(19) United States
(12) Patent Application Publication
(10) Pub. No.: US 2011/0123457 A1
(43) Pub. Date: May 26, 2011

(54) CONJUGATES OF 19F MR IMAGING TRACERS AND CHEMOTHERAPEUTIC AGENTS FOR DRUG QUANTIFICATION AND DRUG DOSE INDIVIDUALIZATION

(76) Inventor: Yihua Yu, Ellicott City, MD (US)

(21) Appl. No.: 12/971,768

(22) Filed: Dec. 17, 2010

Related U.S. Application Data
(63) Continuation-in-part of application No. PCT/US2009/047648, filed on Jun. 17, 2009.
(60) Provisional application No. 61/073,948, filed on Jun. 19, 2008.

(51) Int. Cl. A61K 49/14; A61K 49/10; C07H 19/067; A61P 35/00; C07K 9/00

(52) U.S. Cl. 424/9.34; 424/9.35; 536/28.51; 536/28.55; 530/322

(57) ABSTRACT

A therapeutic agent formed of a magnetic resonance imaging tracer conjugated with a chemotherapeutic agent. The therapeutic agents can be used in measuring drug delivery to a target tissue. The therapeutic agents allow for therapeutic MRI, in which ¹⁹F MRI techniques are used to detect, monitor, evaluate, and/or adjust chemotherapeutic drug dosage levels in a patient or a targeted tissue thereof.

### CROSS REFERENCE TO RELATED APPLICATION

This application is a continuation-in-part of International Patent Application PCT/US2009/047648, filed on 17 Jun. 2009, which claims the benefit of U.S. provisional patent application, Ser. No. 61/073,948, filed on 19 Jun. 2008. The co-pending International Patent Application patent application and the U.S. provisional patent application are hereby incorporated by reference herein in their entireties and are made a part hereof, including but not limited to those portions which specifically appear hereinafter.

### BACKGROUND OF THE INVENTION

This invention relates generally to drug dosing in clinical practice and, more particularly, to a use of imaging technology in the administering and measuring of drugs within a patient.

The dosage of an administered drug is very important to achieve the desired therapeutic effect, while at the same time reducing risks of adverse effects (Donald R. Stanski et al., Getting the Dose Right: Report From the Tenth European Federation of Pharmaceutical Sciences (EUFEPS) Conference on Optimizing Drug Development, J. of Pharmacokinetics and Pharmacodynamics, Vol. 32, No. 2, April 2005, 199-211). The importance of a drug dosage is reflected in Federal rules that require dosage information, such as recommended doses, dose range, dosing intervals, treatment duration, and modifications for special patient populations, to be provided with the drug (James Cross et al., Postmarketing drug dosage changes of 499 FDA-approved new molecular entities, 1980-1999, Pharmacoepidemiology and Drug Safety, 2002; 11: 439-446).

However, it has been determined that over time clinical dosing requirements change from the manufacturer's initial recommendation (Ebert R. Heerdink et al., Changes in prescribed drug doses after market introduction, Pharmacoepidemiology and Drug Safety 2002; 11: 447-453). Dosing changes are often motivated by safety concerns, and the rate of dosing changes is typically greater for newer drugs (James Cross et al., Postmarketing drug dosage changes of 499 FDA-approved new molecular entities, 1980-1999, Pharmacoepidemiology and Drug Safety, 2002; 11; 439-446). It has been estimated that as many as one in five effective prescription drugs must be relabeled or removed from the market as a result of incorrect dosing by practitioners (C. C. Peck et al., “Getting the Dose Right ”: Facts, a Blueprint, and Encouragements, Nature, Vol. 82, No. 1, July 2007, 12-14). Perhaps this is at least partly because recommended dosages are often determined early in a drug development program (H: A. J. Struijker Boudier, A drug is not a drug is not a drug: a commentary, Pharmacoepidemiology and Drug Safety, 2002; 11: 437-438).

It has been estimated that underdosing occurs in about 50% of patients treated with various protocols for different types of cancer (H. Gurney, I don't underdose my patients . . . do I?, The Lancelot Oncology, Vol. 6, September 2005, pp. 637-638). As an example, such underdosing has been determined to affect survival in the treatment of advanced non-small-cell lung cancer (NSCLC) (Id.). The underdosing may be due to the common use of body surface area as the only independent variable in determining dosage, which leads to large interpatient variability and intrapatient variability (at different disease stages) in drug exposure. (Sharyn D. Baker et al., Role of Body Surface Area in Dosing of Investigational Anticancer Agents in Adults, 1991-2001, J. of the Nat. Cancer Inst., Vol. 94, No. 24, Dec. 18, 2002, 1883-1888; Walter J. Loos et al., Inter- and intrapatient Variability in Oral Topotecan Pharmacokinetics: Implications for Body-Surface area Dosage Regimes, Clinical Cancer Research, 6, 2685-2689, 2000).

Therefore, individual dose-titration remains a viable treatment strategy that can account for patient differences (H. A. J. Struijker Boudier, Pharmacoepidemiology and Drug Safety 2002, pp. 437-438). There is an ongoing need for new ways for determining proper dosage and the amount of a drug reaching the targeted tissue.

### SUMMARY OF THE INVENTION

A general object of the invention is to provide a method for determining drug dosages in a target tissue and other vital organs of a patient, as well as compounds for use in implementing the method.

The general object of the invention can be attained, at least in part, through a method of administering a drug treatment to a mammal. The method includes administering to the mammal a dose of a therapeutic agent and measuring an amount of the chemotherapeutic agent (e.g., the treatment drug compound) in a tissue or organ of the mammal using magnetic resonance imaging (MRI).

The therapeutic agent of this invention, and that which is contemplated for use in the above described method, comprises a magnetic resonance imaging (MRI) imaging tracer conjugated with the chemotherapeutic agent.

The therapeutic agent of this invention allows for therapeutic MRI according to this invention. In therapeutic MRI according to this invention, MRI techniques, and particularly ¹⁹F MRI techniques, are used to detect, monitor, measure, evaluate, and/or adjust drug dosage levels in a patient or a targeted tissue or organ thereof.

Therapeutic MRI according to this invention integrates MRI with drug delivery. The therapeutic agent of this invention includes, for example, an anticancer drug, or prodrug thereof, conjugated with an MR imaging tracer, forming a dual pharmaceutical entity for both MRI detection and treatment. Throughout a course of treatment, a patient will receive the therapeutic agent and MRI scans are conducted intermittently to determine the optimal dose for each patient, such as at each treatment stage. The method and therapeutic agent of this invention allow for individualized dosing, and drug doses can, for example, be adjusted depending on the amount of the drug measured by MRI in the target tissue.

MRI is a known technique for obtaining images of the inside of an object under investigation, such as a patient. An MRI apparatus generates a static magnetic field around at least a portion of the object, so as to order or align the random ordered nuclear spins of the nuclei in the object. A radio-frequency (RF) antenna system is also a part of the apparatus, and includes an RF transmission coil and at least one RF reception coil. In some instances, the RF transmission coil and the RF reception coil may be the same. RF energy is irradiated into the examination subject by the RF transmission coil, causing magnetic resonance signals to be generated in the subject, which are detected (received) by the RF reception coil or coils. The received, analog magnetic resonance signals are converted into digital signals, and represent a so-called raw data set. The raw data set is obtained in the Fourier domain, also known as k-space. By means of an inverse Fourier transformation, the data in k-space are transformed into image data.

MRI techniques include the detection of particular atomic nuclei (e.g., those possessing magnetic dipole moments) utilizing the above discussed magnetic fields and radio-frequency radiation. It is similar in some respects to x-ray computed tomography in providing a cross-sectional display of the body organ anatomy, only with excellent resolution of soft tissue detail. However, unlike x-ray computer tomography, MRI does not use ionizing radiation. MRI is, therefore, a safer non-invasive technique for medical imaging.

The hydrogen atom, having a nucleus consisting of a single unpaired proton, has one of the strongest magnetic dipole moments of nuclei found in biological tissues. As hydrogen occurs in both water and lipids, it is abundant in the human body. Therefore, MRI is most commonly used to produce images based upon the distribution density of protons and/or the relaxation times of protons in organs and tissues. Other nuclei having a net magnetic dipole moment also exhibit a nuclear magnetic resonance phenomenon which may be used in MRI applications. Such nuclei include carbon-13 (six protons and seven neutrons), fluorine-19 (9 protons and 10 neutrons), sodium-23 (11 protons and 12 neutrons), and phosphorus-31 (15 protons and 16 neutrons).

In MRI, the nuclei under study in a sample (e.g., ¹⁹F, etc.) are irradiated with the appropriate radio-frequency (RF) energy in a controlled gradient magnetic field. These nuclei, as they relax, subsequently emit RF energy at a sharp resonance frequency. The resonance frequency of the nuclei depends on the applied magnetic field. In some cases, the concentration of nuclei to be measured is not sufficiently high to produce a detectable magnetic resonance signal. Signal sensitivity may be improved by administering higher concentrations of the target nuclei or by coupling the nuclei to a suitable “probe” which will concentrate in the body tissues of interest.

As used herein, the term “chemotherapeutic agent” refers to a chemical compound that is (i.e., drug) or becomes (i.e., prodrug), for example, selectively destructive or selectively toxic to the causative agent of a disease, such as malignant cells and tissues, viruses, bacteria, or other microorganism. Chemotherapeutic agents treat a disease by chemical interactions, as compared to radiotherapy, which relies upon radiation for treating a disease.

As used herein, the term “alkyl” refers to a hydrocarbon group that can be conceptually formed from an alkane, alkene, or alkyne by removing hydrogen from the structure of a cyclic or non-cyclic hydrocarbon compound having straight or branched carbon chains, and replacing the hydrogen atom with another atom or organic or inorganic substituent group. In some aspects of the invention, the alkyl groups are “C₁ to C₆ alkyl” such as methyl, ethyl, propyl, isopropyl, n-butyl, iso-butyl, sec-butyl, tert-butyl, amyl, tert-amyl, and hexyl groups, their alkenyl analogues, their alkynyl analogues, and the like. Many embodiments of the invention comprise “C₁ to C₄ alkyl” groups (alternatively termed “lower alkyl” groups) that include methyl, ethyl, propyl, iso-propyl n-butyl, iso-butyl, sec-butyl, and t-butyl groups, their alkenyl analogues, their alkynyl analogues, or the like. Some of the preferred alkyl groups of the invention have three or more carbon atoms preferably 3 to 16 carbon atoms, 4 to 14 carbon atoms, or 6 to 12 carbon atoms. The alkyl group can be unsubstituted or substituted. A hydrocarbon residue, for example an alkyl group, when described as “substituted,” contains or is substituted with one or more independently selected heteroatoms such as O, S, N, P, or the halogens (fluorine, chlorine, bromine, and iodine), or one or more substituent groups containing heteroatoms (OH, NH₂, NO₂, SO₃H, and the like) over and above the carbon and hydrogen atoms of the substituent residue. Substituted hydrocarbon residues may also contain carbonyl groups, amino groups, hydroxyl groups and the like, or contain heteroatoms inserted into the “backbone” of the hydrocarbon residue. In one aspect, an “alkyl” group can be fluorine substituted. In a further aspect, an “alkyl” group can be perfluorinated.

In certain aspects, the term “alkyl” as used herein is a branched or unbranched saturated hydrocarbon group of 1 to 24 carbon atoms, for example 1 to 12 carbon atoms or 1 to 6 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein. A “lower alkyl” group is an alkyl group containing from one to six carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The terms “amine” or “amino” as used herein are represented by the formula NA¹A²A³, where A¹, A², and A³ can be, independently, hydrogen or substituted or unsubstituted alkyl, cycloalkyl, alkenyl, cycloalkenyl, alkynyl, cycloalkynyl, aryl, or heteroaryl group as described herein.

The term “carboxylic acid” or “carboxyl group” as used herein is represented by the formula —C(O)OH.

The term “ester” as used herein is represented by the formula —OC(O)A¹ or —C(O)OA¹, where A¹ can be a substituted or unsubstituted alkyl, cycloalkyl, alkenyl, cycloalkenyl, alkynyl, cycloalkynyl, aryl, or heteroaryl group as described herein. The term “polyester” as used herein is represented by the formula 1(A¹O(O)C-A²-C(O)O)ₐ— or -(A¹O(O)C-A²-OC(O))ₐ—, where A¹ and A² can be, independently, a substituted or unsubstituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “hydroxyl” as used herein is represented by the formula —OH.

The term “silyl” as used herein is represented by the formula —SiA¹A²A³, where A¹, A², and A³ can be, independently, hydrogen or a substituted or unsubstituted alkyl, cycloalkyl, alkoxy, alkenyl, cycloalkenyl, alkynyl, cycloalkynyl, aryl, or heteroaryl group as described herein.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Other objects and advantages will be apparent to those skilled in the art from the following detailed description taken in conjunction with the appended claims.

### BRIEF DESCRIPTION OF THE DRAWINGS

FIG. 1 illustrates a representative ¹⁹F therapeutic agent according to one embodiment of this invention.

FIG. 2 illustrates a representative ¹⁹F therapeutic agent according to another embodiment of this invention.

FIG. 3 illustrates a representative therapeutic agent according to yet another embodiment of this invention.

### DETAILED DESCRIPTION OF THE INVENTION

The present invention provides therapeutic agents, and pharmaceutical compositions thereof, that can be used in drug delivery to a target tissue, and which are also, or include, magnetic resonance imaging (MRI) detectable imaging tracers. The therapeutic agent of this invention allows for therapeutic MRI according to this invention. In the therapeutic MRI methods according to this invention, MRI techniques are used to detect, monitor, evaluate, and/or adjust drug dosage levels in a patient or a targeted tissue thereof.

Before the present compounds, compositions, and/or methods are disclosed and described, it is to be understood that they are not limited to specific synthetic methods unless otherwise explicitly stated, or to particular reagents unless otherwise explicitly stated, as such may, of course, vary. It is also to be understood that the terminology used herein is for the purpose of describing particular embodiments only and is not intended to be limiting.

In one embodiment of this invention, the therapeutic agent includes an MR imaging tracer conjugated with a chemotherapeutic agent. The therapeutic agent and the use thereof in the methods of this invention are not limited to the use of any particular imaging tracer. Various and alternative known and available MRI imaging tracers, including both positive and negative imaging tracers, are suitable for use in the therapeutic agent and methods of this invention. Exemplary imaging tracers include, without limitation, compounds containing as their active element fluorine, as well as gadolinium, manganese, or iron.

In one particularly preferred embodiment of this invention, the suitable imaging tracer is or includes a fluorocarbon. A desirable fluorocarbon imaging tracer includes at least one, and preferably a plurality of, flourine-19 (¹⁹F) nuclei, which are detectable by ¹⁹F MRI. Naturally occurring fluorine atoms (¹⁹F) generally provide a clear nuclear magnetic resonance signal, and thus can function as imaging tracers or passive markers in MRI. Particular benefits of using ¹⁹F include: 1) an extremely low endogenous concentration in the body (fluorine is not naturally found in the body), 2) a high nuclear magnetic resonance sensitivity, and 3) a magnetogyric ratio close to that of ¹H, thus permitting ¹⁹ F magnetic resonance imaging to be carried out with only minor modifications of existing MRI equipment.

In one embodiment of this invention the imaging tracer for us in conjugation to form the therapeutic agent of this invention comprises a compound including the structure:

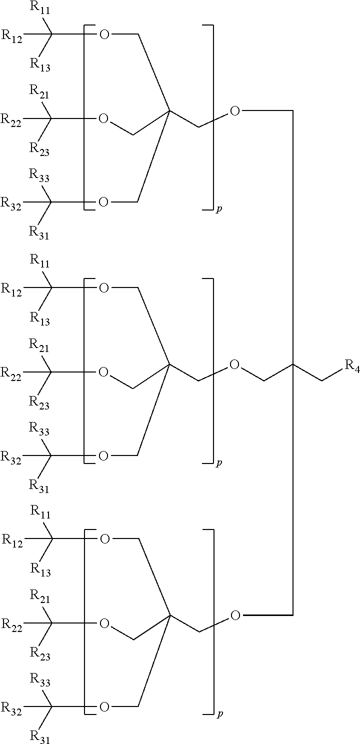

where p is a non-negative integer; R₁₁, R₁₂, R₁₃, R₂₁, R₂₂, R₂₃, R₃₁, R₃₂, and R₃₃ are, independently, H, CH₃, CF₃, or alkyl; and R₄ is H, OH, OBn, OC(CF₃)₃, alkyl, or alkoxy. In a further embodiment, p is 2, 3, 4, or 5. In one embodiment, at least one of R₁₁, R₁₂, R₁₃, R₂₁, R₂₂, R₂₃, R₃₁, R₃₂, or R₃₃ is CF₃. In a further embodiment, R₁₁, R₁₂, R₁₃, R₂₁, R₂₂, R₂₃, R₃₁, R₃₂, and R₃₃ are CF₃.

In a yet further embodiment, the imaging tracer comprises a compound including the structure:

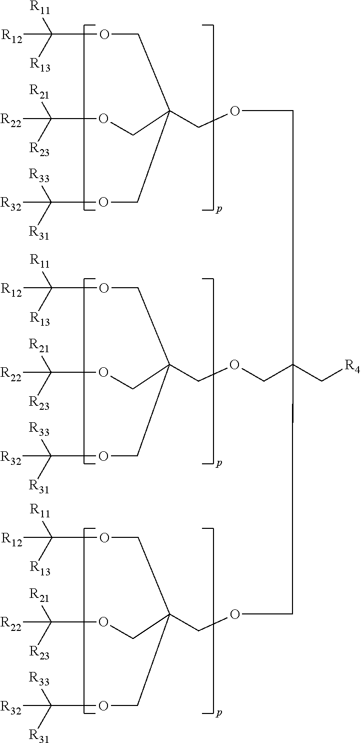

where R₁₁, R₁₂, R₁₃, R₂₁, R₂₂, R₂₃, R₃₁, R₃₂, and R₃₃ are, independently, H, CH₃, CF₃, or alkyl; and R₄ is H, OH, OBn, OC(CF₃)₃, alkyl, or alkoxy. In one aspect, at least one of R₁₁, R₁₂, R₁₃, R₂₁, R₂₂, R₂₃, R₃₁, R₃₂, or R₃₃ is CF₃. In a further aspect, R₁₁, R₁₂, R₁₃, R₂₁, R₂₂, R₂₃, R₃₁, R₃₂, and R₃₃ are CF₃.

One particularly preferred imaging tracer comprises the structure:

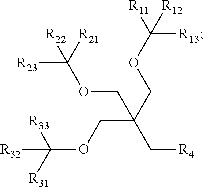

where R₁₁, R₁₂, R₁₃, R₂₁, R₂₂, R₂₃, R₃₁, R₃₂, and R₃₃ are, independently, H, CH₃, CF₃, or alkyl, and R₄ is H, OH, OBn, OC(CF₃)₃, alkyl, or alkoxy. In a further embodiment, R₁₁, R₁₂, and R₁₃ are CF₃. In a further embodiment, R₂₁, R₂₂, and R₂₃ are CF₃. In a further embodiment, R₃₁, R₃₂, and R₃₃ are CF₃. In one embodiment, at least one of R₁₁, R₁₂, R₁₃, R₂₁, R₂₂, R₂₃, R₃₁, R₃₂, or R₃₃ is CF₃. In a further aspect, R₁₁, R₁₂, R₁₃, R₂₁^{,} R₂₂, R₂₃, R₃₁, R₃₂, and R₃₃ are CF₃.

The therapeutic agent including the imaging tracers described above is desirably conjugated at or through the R₄ position to a drug or a prodrug, particularly a chemotherapeutic agent of this invention. The chemotherapeutic agent or agents conjugated to the imaging tracer at or through the R₄ position is not intended to be limited to any particular chemotherapeutic agent. In one preferred embodiment, the chemotherapeutic agent comprises a prodrug that will be converted to an active drug form in vivo.

It will be appreciated by those skilled in the art following the teachings herein provided, that while the chemotherapeutic agent can be an active drug form or a prodrug form, the conjugation of an active drug to an imaging tracer of this invention results in the therapeutic agent of this invention being a prodrug itself.

In one embodiment of this invention, the therapeutic agent includes an MRI imaging tracer that is conjugated to a plurality of chemotherapeutic agents. The MR imaging tracer includes at the R₄ position, a branching module including a plurality of branching units. Each of the branching units is conjugated to one of the plurality of chemotherapeutic agents. An exemplary branching module comprises iminodicarboxylic acid.

In one embodiment of this invention, R₄ of any of the above imaging tracers comprises the structure:

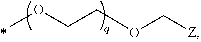

where q is a non-negative integer (such as 0-3), and Z comprises a chemotherapeutic agent or a substituted or unsubstituted amide. One exemplary substituted or unsubstituted amide comprises the structure:

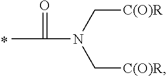

where each R is OH, NH₂, NH-alkyl, alkyl, a polyalkylene oxide, or further conjugated to the chemotherapeutic agent. In another embodiment, the substituted or unsubstituted amide comprises the iminocarboxylic acid structure:

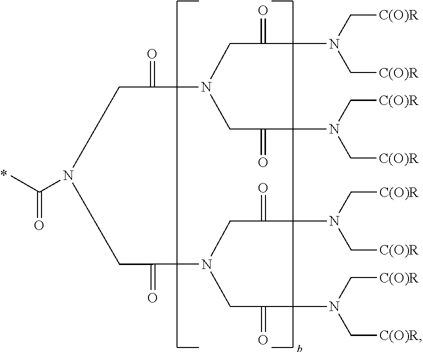

wherein b is a non-negative integer, and each R is OH, NH₂, NH-alkyl, alkyl, a polyalkylene oxide, or further conjugated to the chemotherapeutic agent. In an exemplary embodiment, b iso, 1, 2, or 3.

The therapeutic agent of this invention can optionally include a hydrophilicity enhancing module connecting each of the plurality of branching units to the corresponding one of the plurality of chemotherapeutic agents. Referring to the branching units described above, a hydrophilicity enhancer can be attached at the R position. Desirably, the therapeutic agent includes a hydrophilicity enhancing module connecting each of the plurality of branching units to one of the plurality of chemotherapeutic agents of the therapeutic agent. Hydrophilicity enhancing modules according to this invention help ensure rapid renal excretion of the imaging tracer after the chemotherapeutic agent is cleaved in vivo from the therapeutic agent (discussed further below). An exemplary hydrophilicity enhancer for use according to this invention is an oligo-oxyethylene.

In one embodiment of this invention, each imaging tracer contains multiple conjugation sites that can each be used to covalently link the imaging tracer to other molecules, such as drugs, prodrugs, ¹H MR imaging agents, proteins, antibodies, etc.

Examples of prodrugs and chemotherapeutic agents that can be coupled to ¹⁹F imaging agents as described herein include and are not are not limited to: bambuterol (prodrug for terbutaline), clopidogrel, analapril, pivampicillin, ximelagatran, famciclovir, tenofovir disoproxil, adefovir dipivoxil, oseltamivir, prednisolone, fludarabine, estramustine, miproxifene, propofol, irinotecan, valacyclovir, valganciclovir, midodrine, dipivefrin, latanoprost, tazarotene, levodopa, pradefovir, simvastatin, octreotide, sulindac, albendazole, clindamycin phosphate, etoposide, aminodarone, parecoxib, oxazepam, oxazepam, promedrol. Other examples of prodrugs and chemotherapeutic agents that can be employed in the present imaging compositions and methods are described, e.g., in Rautio J. et al., Prodrugs: Design and Clinical Application, Nature Reviews (2008) 7:255-270 (see, e.g., Tables 1-6); Ettmayer P. et al., Lessons Learned from Marketed and Investigational Prodrugs, J. Med. Chem. (2004) 47:2393-2404; Testa B., Prodrug Research: Futile or Fertile? Biochem. Pharmacol. (2004) 68: 20097-2106; Stella V. J. et al., Prodrug Strategies to Overcome Poor Water Solubility, Adv. Drug Delivery Rev. (2007) 59:677-694; Rooseboom M., Enzyme-Catalyzed Activation of Anticancer Prodrugs, Pharmacol. Rev. (2004) 56:53-102, each of which is herein incorporated by reference in its entirety. The skilled artisan will appreciate that any suitable prodrug or chemotherapeutic agent can be coupled to the imaging agents described herein.

To systematically denote the structures of, for example, ¹⁹F imaging tracers (¹⁹FIT), the notation ¹⁹FIT(m, n) can be used, where m refers to the number of fluorine atoms in the ¹⁹FIT and n refers to the number of conjugations sites in ¹⁹FIT. n is considered the valency of ¹⁹FIT. For illustration purposes, the following is the structure of a representative ¹⁹FIT(27, 4):

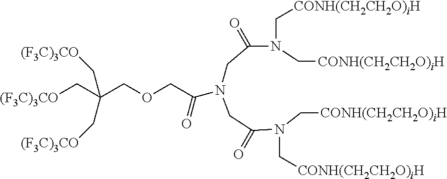

It is important to note that there can be variations within each notation ¹⁹FIT(m, n). For example, in the above ¹⁹FIT(27, 4) the length i of the (—CH₂CH₂O—)ᵢ segment can vary (i is a positive integer such as 1, 2, 3, 4, 5, 6 . . . ).

The following structures represent exemplary therapeutic agents according to one embodiment of this invention, where X is a chemotherapeutic agent. The structures below have the illustrated imaging tracer, branching unit and/or hydrophilicity enhancing module varied according to the above descriptions.

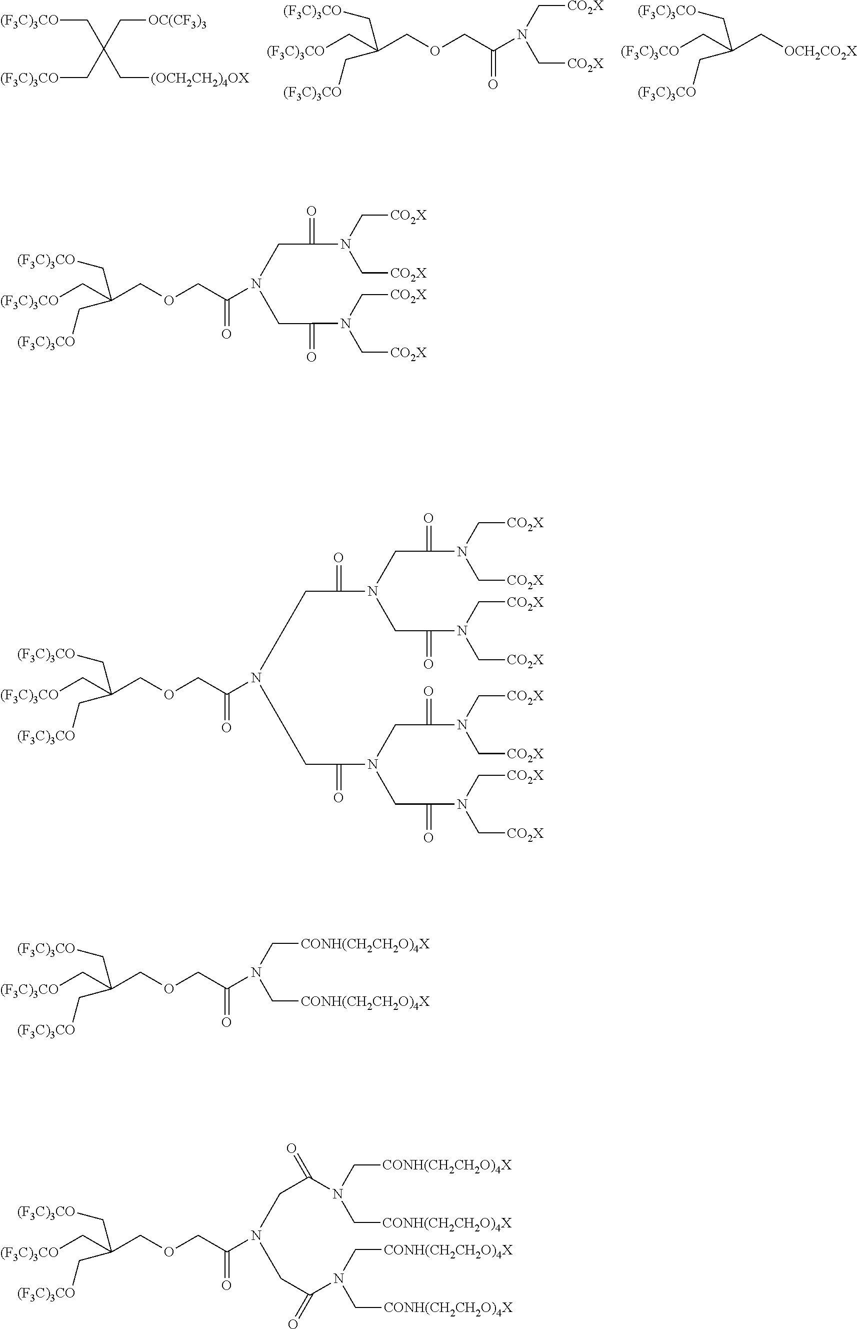

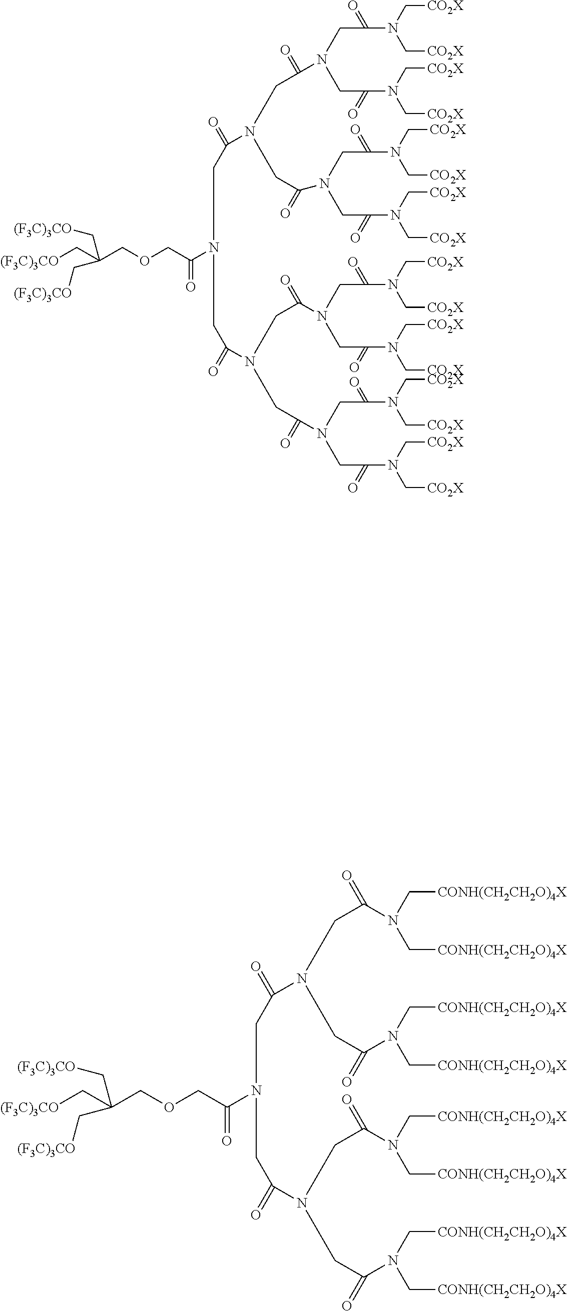

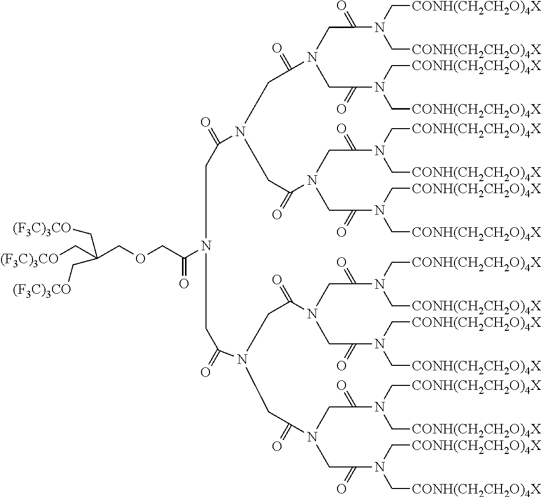

An exemplary process for the preparation of an imaging tracer having the structure:

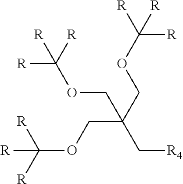

where R is H, CH₃, CF₃, or alkyl and wherein R₄ is H, OH, OBn, alkyl, or alkoxy, includes the steps of: providing a triol, and reacting the triol with tert-butanol or nonafluoro-tert-butanol to provide a tri-tert-butyl ether or a triperfluoro-tert-butyl ether. The reacting step can be performed with nonafluoro-tert-butanol. The triol can be pentraerythritol, mono-silylated pentraerythritol, or 2,2-bis-hydroxymethyl-propan-1-ol. The providing step can be performed by the steps of: mono-protecting pentraerythritol before the reacting step, and deprotecting the product of the reacting step. The reacting step can occur before the deprotecting step. Also, the process can further include the step of coupling the product the deprotecting step with a hydrophilic compound, such as a moiety having the structure:

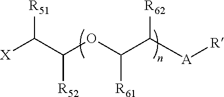

where n is 0 or a positive integer; R₅₁, R₅₂, R₆₁, and R₆₂ are, independently, H or alkyl; R′ comprises H, CH₂CO₂H, silyl, or alkyl; A is O, S, or amino; and X is a leaving group. n can be an integer from 4 to 12. Also, the process can include the step of cleaving the silyl group. The process can further include the step of conjugating with cyclen or a compound comprising a cyclen residue.

The following Schemes 1-4 illustrate exemplary reactions to obtain suitable ¹⁹F imaging tracers (including the fluorocarbon module, branching unit, and hydrophilicity enhancer) for further conjugation with a chemotherapeutic agent according to this invention.

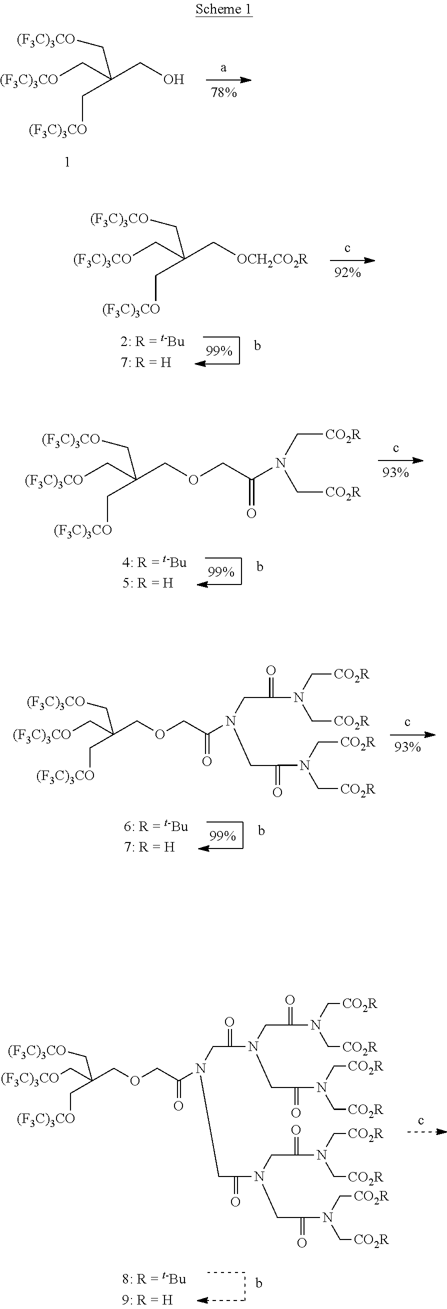

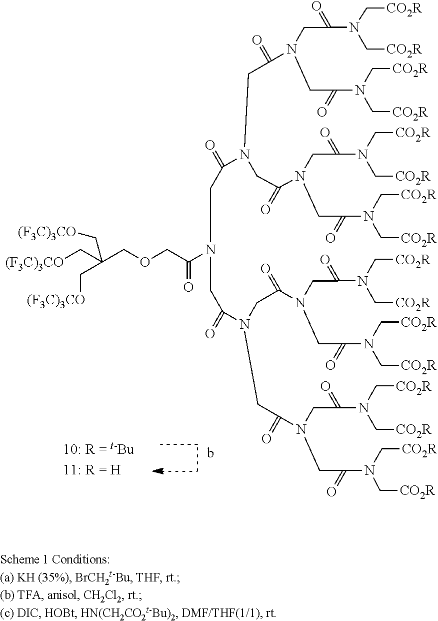

Treatment of alcohol 1 with potassium hydride and tert-butyl bromoacetate gives ester 2 after simple phase separation of the quenched reaction mixture. Ester 2 reacted with trifluoroacetic acid gives the acid 3 after removal of reaction solvent, anisol, and TFA. Acid 3 is coupled with di-tert-butyl iminodiacetate to yield ester 4 after fluorous solid phase extraction. By repeating the coupling and deprotecting processes, the further branched intermediates are obtained.

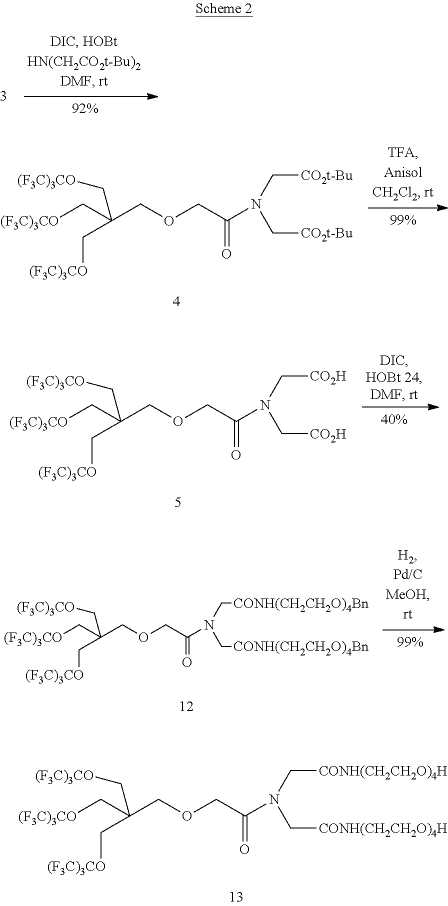

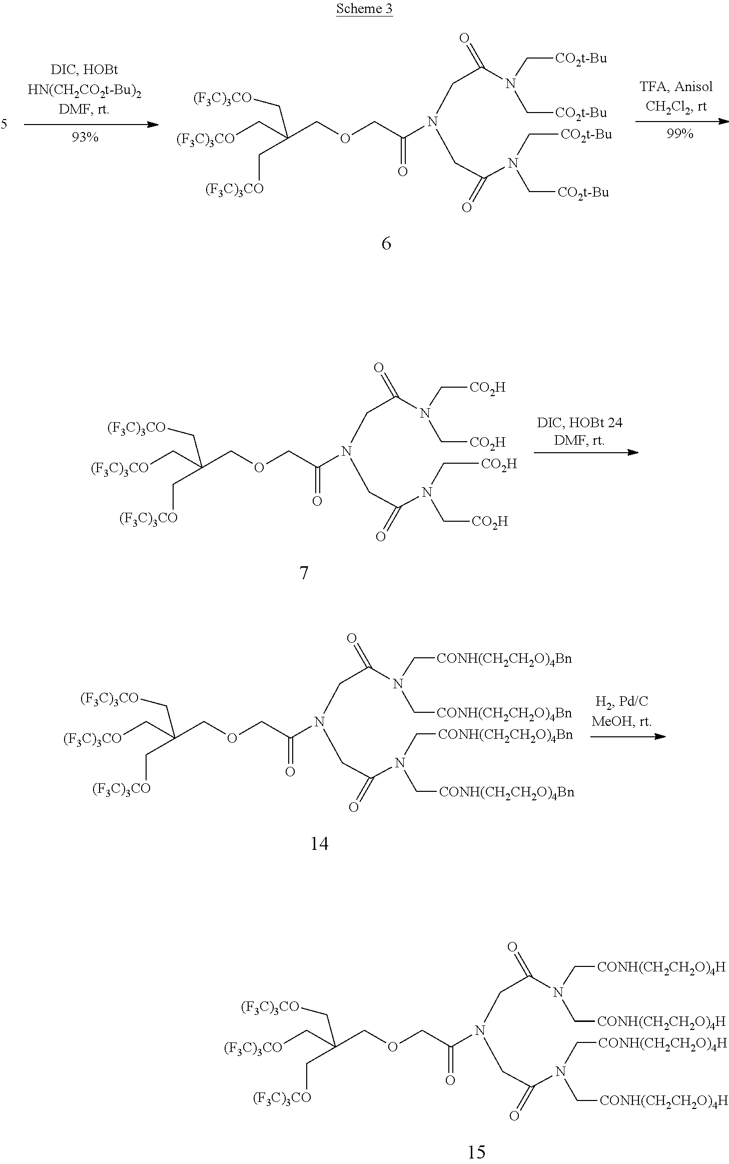

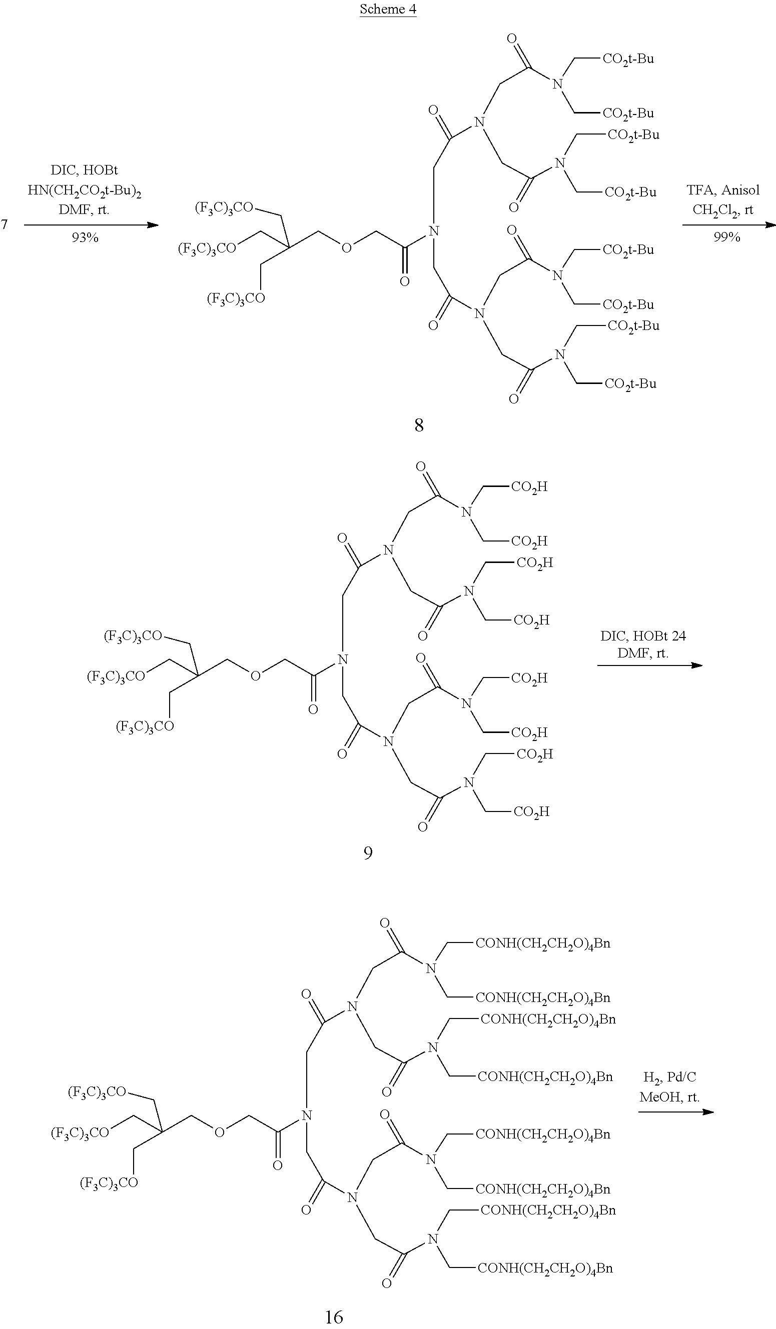

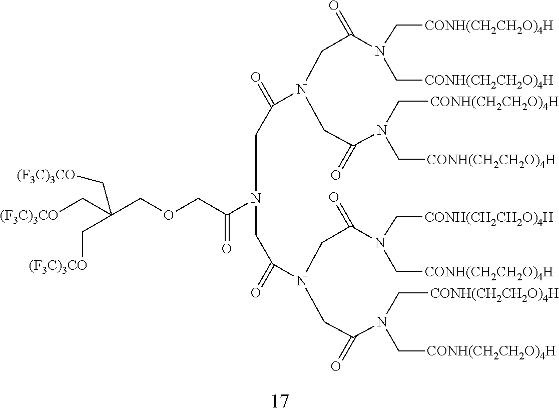

As mentioned above, the therapeutic agent of one embodiment of this invention includes, as the chemotherapeutic agent conjugated to the imaging tracer, a prodrug that will covert to an active, free drug form within the patient, and desirably within the targeted tissue or cells. One benefit of the therapeutic agent of this invention is to allow the pharmacokinetics and tissue concentrations of the delivered prodrug or drug to be quantified by MR right up to the point where the therapeutic agent is converted to the free drug. The therapeutic agent to free drug conversion process can also thus be monitored by MR, such as by magnetic resonance spectroscopy.

One such suitable prodrug, as an example, is capecitabine (CAP), an anticancer drug (Xeloda®). CAP is enzymatically converted to its active cancer drug form, 5-fluorouracil (5-FU), in three steps (CAP→5′-DFCR→5′-DFUR→5-FU), as shown below, with the last step catalyzed by thymidine phosphorylase (TP), preferably within a tumor. According to this invention, the CAP prodrug can be conjugated to one of the ¹⁹F imaging tracers (¹⁹FIT) discussed above to prepare a therapeutic agent (¹⁹FIT-CAP) that will be converted to 5-FU in three steps similar to that of CAP (¹⁹FIT-CAP→¹⁹FIT-5′-DFCR→¹⁹FIT-5′-DFUR→5-FU).

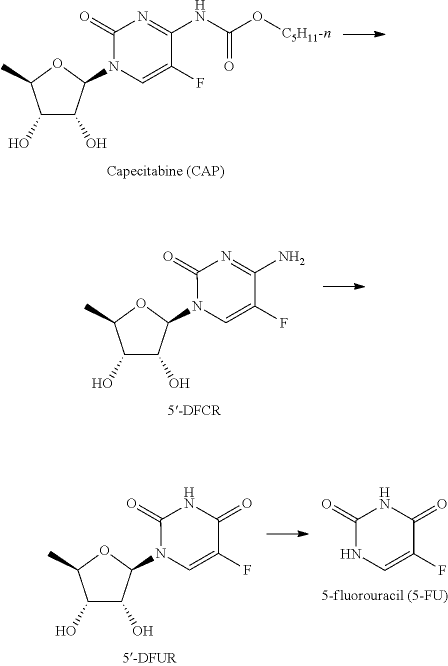

The following are several exemplary structures (not exhaustive) of ¹⁹FIT-CAP prodrugs according to one embodiment of this invention.

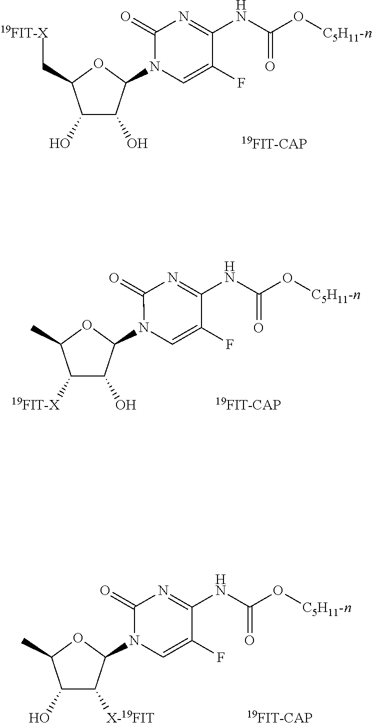

In the above structures, X is a linker group between ¹⁹FIT and CAP. The identity of the linker group depends on the conjugation chemistry between ¹⁹FIT and CAP, which can be an amide bond, ether bond, thiol-ether bond, ester bond, etc. For example, for an amide bond conjugation, X═—CO—NH—; and for an ether bond conjugation, X═—O—. ¹⁹FIT can be conjugated in similar fashion to 5′-DFCR and 5′-DFUR to form prodrugs of 5-FU. The ¹⁹FIT can be attached to the pro-moiety of existing pro-drugs of other drugs in addition to 5-FU.

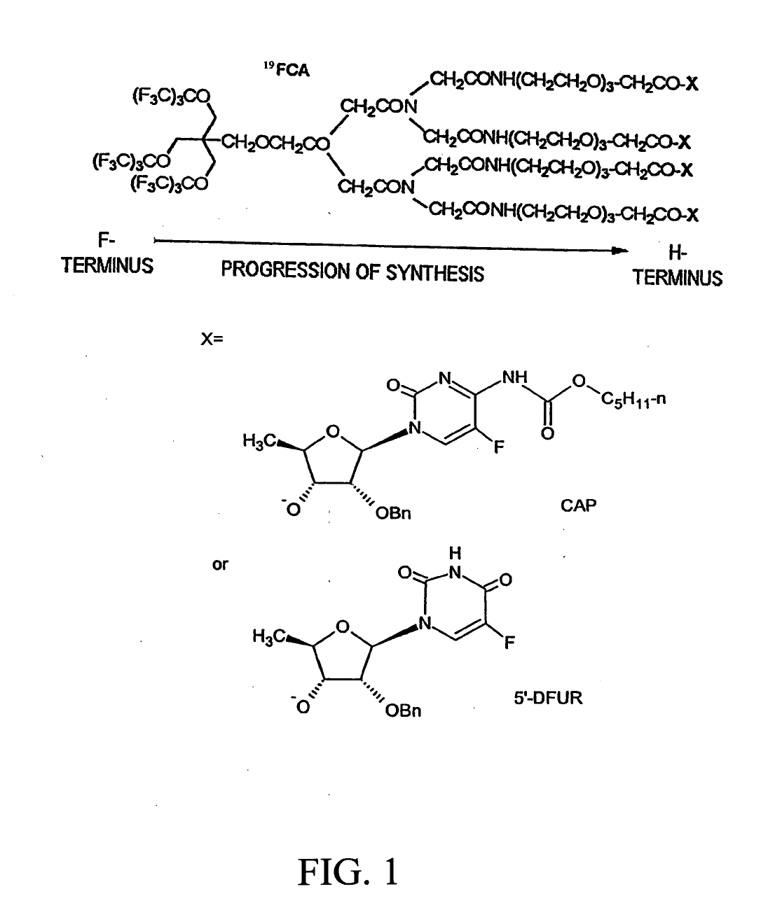

In an embodiment where the ¹⁹FIT comprises three modules, namely a fluorocarbon module, a branching unit module, and a hydrophilicity enhancing module (such as shown in FIG. 1 as an example), the ¹⁹FIT can be assembled via peptide bonds from the F-terminus to the H-terminus, analogous to peptide synthesis. Each of the identical H-terminus carboxylic groups (at —X below) is then conjugated to CAP. In an alternative embodiment, the 5′-DFUR prodrug is substituted for the CAP prodrug. CAP and 5′DFUR each contain two hydroxyl groups. During conjugation, one hydroxyl group is protected by, for example, a benzyl (Bn) group, and the other hydroxyl group is the conjugate takes place. After conjugation, the benzyl protecting group is cleaved off.

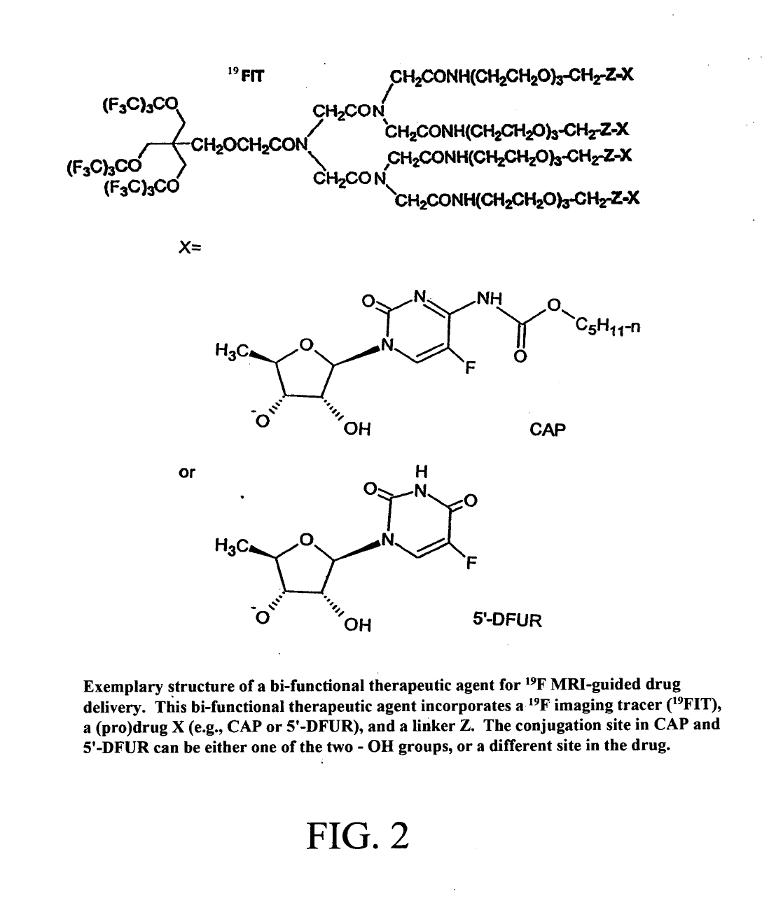

The conjugation sites in both the chemotherapeutic agent and ¹⁹FIT can be derivatized into —SH, —NH2, —COOH, etc. (various combinations) for the conjugation reaction. The conjugation may involve, but is not restricted to, the formation of (thio)ether bonds, (thio)ester bonds, amide bonds, C—C bonds, etc. An exemplary structure (the number of branches in ¹⁹FIT can vary) of the resulting bi-functional therapeutic agent is shown in FIG. 2.

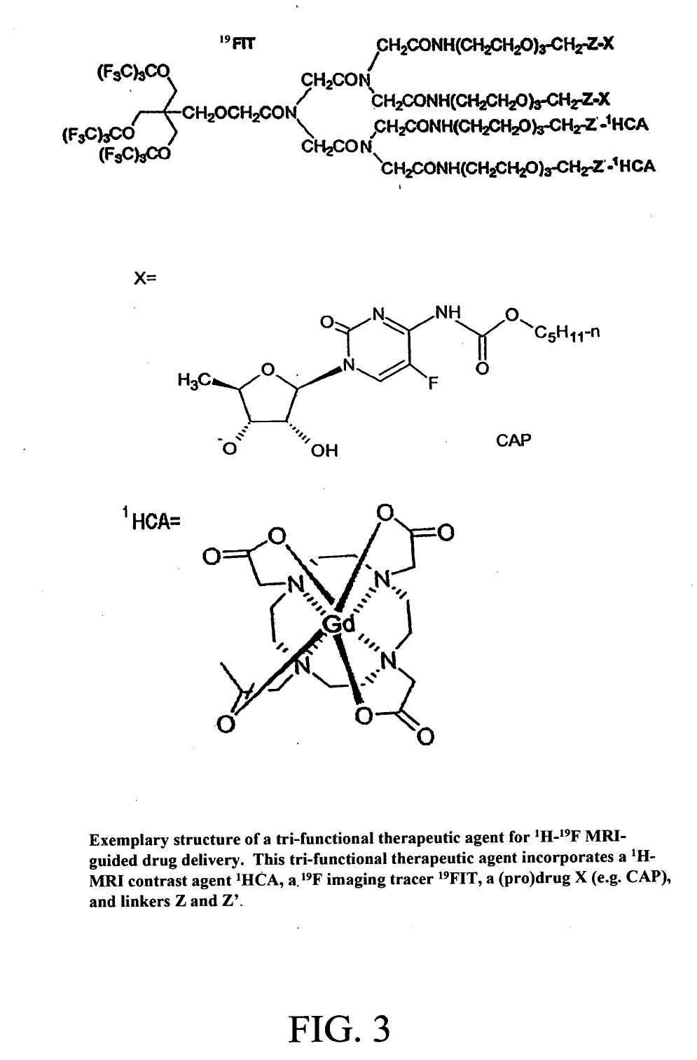

In another embodiment of this invention, the therapeutic agent can include more than one type of imaging tracer, thereby forming a tri-functional therapeutic agent. Additional imaging tracers that can be conjugated to ¹⁹FIT are discussed above. As an example, a Gd(III)-based contrast agent can be conjugated to ¹⁹FIT, forming the dual-nuclei imaging agent ¹HCA-¹⁹FIT. This dual nuclei imaging agent can be conjugated to the chemotherapeutic agent X of this invention via a linker group Z (see FIGS. 2 and 3), such as to allow for MR imaging of a tissue as well as drug quantification according to this invention. As a result, ¹HCA-¹⁹FIT-X is formed. The benefit of this embodiment is that while ¹H MRI is suited for providing body information, ¹⁹F is suited for providing drug information. Together, they can provide a comprehensive picture of the body and the drug for dose individualization applications.

The structure in FIG. 3 illustrates an exemplary structure (e.g., the number of branches in ¹⁹FIT is variable (4 branches below)) of a tri-functional therapeutic agent according to this invention. The chelator used in ¹HCA (contrast agent) is not restricted to DOTA, as shown below. The chemotherapeutic agent can be any prodrug or drug moiety. The molar ratio of X/¹HCA is variable (=1 in this rendering). The arrangement and position of X and ¹HCA can also be changed. The linker group Z and Z′ may or may not be identical.

The present invention also provides a pharmaceutical composition comprising a pharmaceutically acceptable carrier and a therapeutic agent of this invention. Suitable pharmaceutically acceptable carriers described herein, for example, vehicles, adjuvants, excipients, and diluents, are well-known to those skilled in the art and are readily available to the public. The choice of carrier will be determined, in part, by the particular composition and by the particular method used to administer the composition. Accordingly, there are a wide variety of suitable formulations of the pharmaceutical compositions of the present invention.

The present invention also relates to method of treating diseases or conditions, such as a disease of uncontrolled cellular proliferation, including, without limitation, carcinoma, lymphoma, leukemia, or sarcoma or other cancers and tumors, by administering to a subject in need thereof an effective amount of a therapeutic agents compound in accordance with the present invention. The term “treating” is used conventionally, e.g., the management or care of a subject for the purpose of combating, alleviating, reducing, relieving, improving, etc., one or more of the symptoms associated with the disease. The treatment can be prophylactic or therapeutic. “Prophylactic” refers to any degree in inhibition of the onset of a cellular disorder, including complete inhibition, such as in a patient expected to soon exhibit the cellular disorder. “Therapeutic” refers to any degree in inhibition or any degree of beneficial effects on the disorder in the mammal (e.g., human), e.g., inhibition of the growth or metastasis of a tumor.

One skilled in the art will appreciate that suitable methods of administering a therapeutic agent of the present invention to an animal, e.g., a mammal such as a human, are known. Although more than one route can be used to administer a particular composition, a particular route can provide a more immediate and more effective result than another route.

Formulations suitable for oral administration can consist of (a) liquid solutions, such as an effective amount of a therapeutic agent of this invention dissolved in a diluent, such as water or saline, (b) capsules, sachets or tablets, each containing a predetermined amount of the active ingredient, as solids or granules, (c) suspensions in an appropriate liquid, and (d) suitable emulsions.

Tablet forms can include one or more of lactose, mannitol, cornstarch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically acceptable and compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, such carriers as are known in the art.

The therapeutic agent of this invention, alone or in combination with other suitable components, can be made into aerosol formulations to be administered via inhalation. These aerosol formulations can be placed into pressurized acceptable propellants, such as dichlorodifluoromethane, hydrofluorocarbon (such as HFC 134a and/or 227), nitrogen, and the like.

Formulations suitable for parenteral administration include aqueous and non-aqueous solutions, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried condition requiring only the addition of the sterile liquid carrier, for example, water, for injections, immediately prior to use. Extemporaneous injection solutions and suspensions can be prepared from sterile powders, granules, and tablets of the kind previously described.

The dose administered to an animal, particularly a human, in the context of the present invention should be sufficient to affect a therapeutic response in the animal over a reasonable time frame. The specific dose level and frequency of dosage may vary, depending upon a variety of factors, including the activity of the specific active compound, its metabolic stability and length of action, rate of excretion, mode and time of administration, the age, body weight, health condition, gender, diet, etc., of the subject, and the severity of, for example, the cancer. Any effective amount of the compound can be administered, e.g., from about 1 mg to about 500 mg per day, about 50 mg to about 150 mg per day, etc. In one embodiment of this invention, a suitable dosage for internal administration is 0.01 to 100 mg/kg of body weight per day, such as 0.01 to 35 mg/kg of body weight per day or 0.05 to 5 mg/kg of body weight per day. A suitable concentration of the compound in pharmaceutical compositions for topical administration is 0.05 to 15% (by weight), preferably 0.02 to 5%, and more preferably 0.1 to 3%. The prodrugs of this invention can be administered in such dosages in any form by any effective route, including, e.g., oral, parenteral, enteral, intraperitoneal, topical, transdermal (e.g., using any standard patch), ophthalmic, nasally, local, non-oral, such as aerosal, spray, inhalation, subcutaneous, intravenous, intramuscular, buccal, sublingual, rectal, vaginal, intra-arterial, and intrathecal, etc.

As discussed above, the therapeutic agents of the present invention can be administered alone, or in combination with any ingredient(s), active or inactive, such as with a pharmaceutically acceptable excipient, carrier or diluent. The therapeutic agents of this invention can also be used in combination with other cancer treatments and drugs. For example, the therapeutic agents of this invention can be used as a part of or in combination with known cancer treatments such as hormone therapy, radiation therapy, immunotherapy, and/or surgery.

The therapeutic agents,of this invention can be administered to a patient at any time as determined by the treating physician. Preferably, the therapeutic agents of this invention are administered for treating a patient during one or more of Stages II-IV of the cancer.

¹⁹F is the second most sensitive nucleus for MR imaging with a sensitivity of 83% of that of ¹H. ¹⁹F imaging is suitable for measuring drug concentration in a mammal according to this invention because there is no detectable background ¹⁹F signal in the mammalian body. The invention includes a method of administering a drug treatment to a mammal, whereby a dose of a therapeutic agent of this invention is administered to the mammal, and an amount of the chemotherapeutic agent provided by the therapeutic agent dose in a tissue or organ of the mammal is measured using ¹⁹F MRI. In one embodiment, the tissue or organ is or includes a tumor or other disease of uncontrolled cellular proliferation.

As discussed above, the chemotherapeutic agent is desirably cleaved from the imaging tracer of the therapeutic agent within the tissue or organ targeted for treatment. Where the chemotherapeutic agent is a prodrug, such as CAP, the cleaving of the imaging tracer desirably occurs during conversion of the prodrug to the active drug form. The measured amount of imaging tracer in the target tissue or organ is indicative of the amount of the administered chemotherapeutic agent that has also reached the target tissue or organ. The therapeutic agents of this invention thus can provide individualized treatment plans by obtaining patient-specific pharmacokinetic information. This is accomplished by tracing the drug surrogate via ¹⁹F MRI and optionally visualizing the tumor via ¹H MR imaging. The aim is to determine whether tumor targeting is achieved and whether the overall biodistribution of the chemotherapeutic agent is acceptable.

The ¹⁹F MR imaging capacity of the imaging tracer of the therapeutic agent of this invention allows a physician to monitor the chemotherapeutic drug directly in real time. Such real time feedback makes it possible to adjust treatment plans immediately. By determining the amount of a particular dose of therapeutic agent that has reached the target tissue or organ, further dosages for the patient can be determined. In one embodiment of this invention, a plurality of doses of the therapeutic agent are given to the mammal, and a plurality of ¹⁹F MRI measurements are conducted of the amount of the chemotherapeutic agent in the tissue or organ of the mammal using ¹⁹F MRI. The measurements are desirably are conducted intermittently, such as one after each dose, and can be used to determine an optimal dose for the mammal using the obtained measurements. The dosage can then be adjusted based upon actual, patient-specific biodistribution measurements. For example, when a first dose is determined to not provide the desired level of chemotherapeutic agent in the target tissue or organ, a second dose can be administered to increase the amount of chemotherapeutic agent in the target tissue or organ, which is again measurable by the ¹⁹F MRI.

During the post-therapy assessment stage (Stage 3), the imaging tracer can be used to visualize the residual tumor (via ¹H-¹⁹F MR imaging) and evaluate its hypoxic status (via ¹⁹F MR relaxometry). Such information helps to determine whether the previous round of therapy is effective and whether another round of therapy is needed. If another round of therapy is needed, the post-therapy assessment stage of the previous round automatically becomes the pre-therapy planning stage of the next round.

Thus, the invention provides a therapeutic agent and method of use that integrates MRI with drug delivery. Throughout a course of treatment, a patient will receive the therapeutic agent, and MRI scans are conducted intermittently to determine the optimal dose for each patient, such as at each treatment stage. The method and therapeutic agent of this invention allow for individualized dosing, and drug doses can, for example, be adjusted depending on the amount of the drug measured by MRI in the target tissue.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A composition, comprising a ¹⁹F magnetic resonance (MR) imaging tracer conjugated with a chemotherapeutic agent, wherein the composition is detectable by ¹⁹F MRI.

2. The composition according to claim 1, wherein the MR imaging tracer comprises a fluorocarbon.

3. The composition according to claim 1, further comprising a dual-nuclei imaging agent.

4. The composition according to claim 1, wherein the chemotherapeutic agent comprises a prodrug.

5. The composition according to claims 1, wherein the MR imaging tracer is conjugated to a plurality of chemotherapeutic agents, the MR imaging tracer comprises a branching module including a plurality of branching units, and each of the branching units is conjugated to one of the plurality of chemotherapeutic agents.

6. The composition according to claim 5, wherein the branching module comprises iminodicarboxylic acid.

7. The composition according to claim 5, wherein the MR imaging tracer comprises a hydrophilicity enhancing module connecting each of the plurality of branching units to the one of the plurality of chemotherapeutic agents.

8. The composition according to claim 7, wherein the hydrophilicity enhancing module comprises oligo-oxyethylene.

9. The composition according to claim 1, comprising the structure: 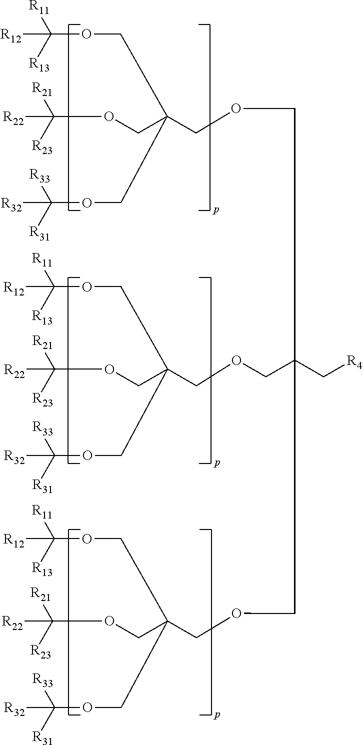
where p is a non-negative integer; each of R₁₁, R₁₂, R₁₃, R₂₁, R₂₂, R₂₃, R₃₁, R₃₂, and R₃₃ is, independently, H, CH₃, CF₃, or alkyl; and R₄ is or comprises the chemotherapeutic agent.

10. The composition according to claim 9, wherein each of R₁₁, R₁₂, R₁₃, R₂₁, R₂₂, R₂₃, R₃₁, R₃₂, and R₃₃ is CF₃.

11. The composition according to claim 9, wherein R₄ comprises the structure: 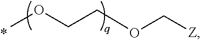
where q is a non-negative integer, and Z comprises the chemotherapeutic agent or a substituted or unsubstituted amide conjugated with the chemotherapeutic agent.

12. The composition according to claim 11, wherein the amide comprises the structure: 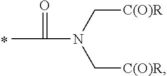
where at least one R comprises the chemotherapeutic agent.

13. The composition according to claim 11, wherein the amide comprises the iminocarboxylic acid structure: 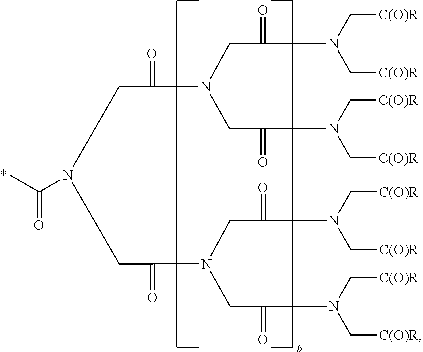
wherein b is a non-negative integer, and at least one R comprises the chemotherapeutic agent.

14. The composition according to claim 9, comprising the structure: 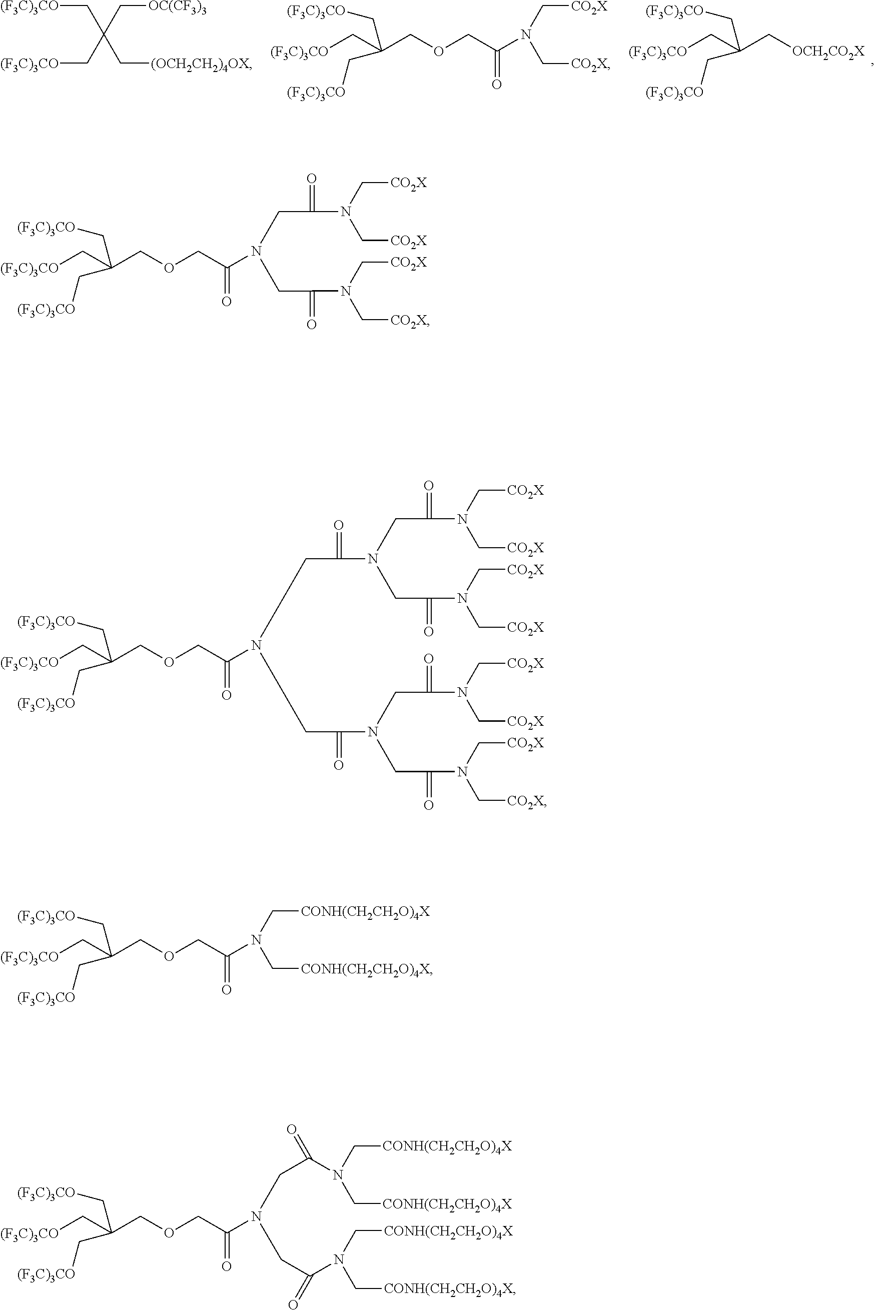 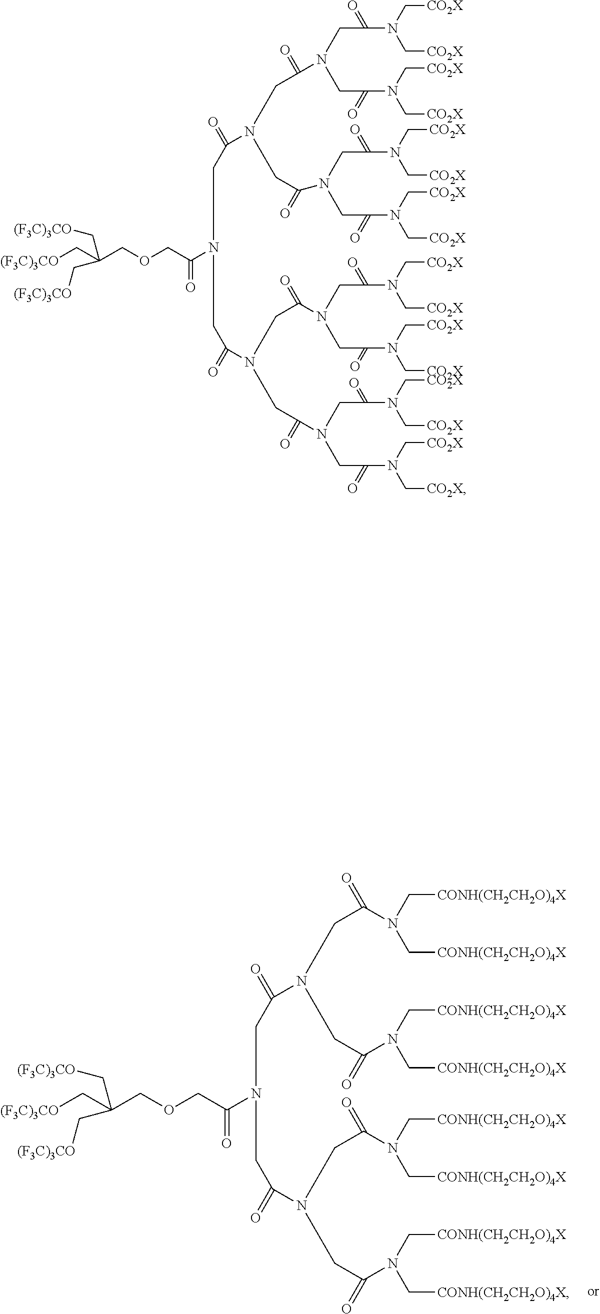 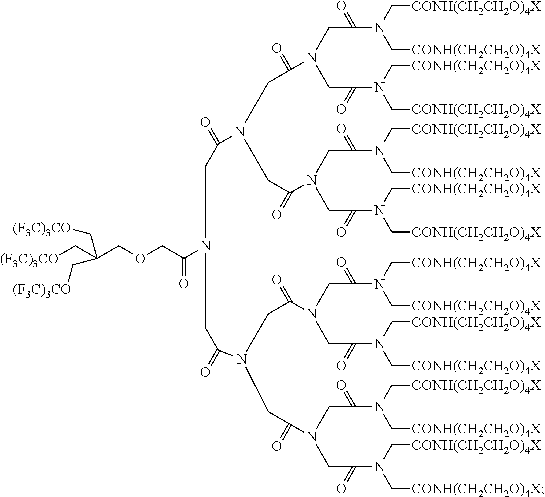
where X is the chemotherapeutic agent.

15. The composition according to claim 9, wherein X comprises:

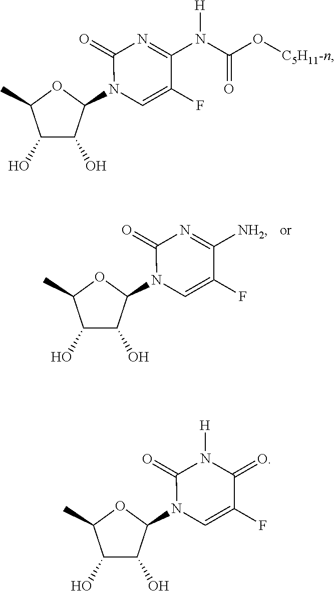

16. The composition according to claim 1, comprising the structure: 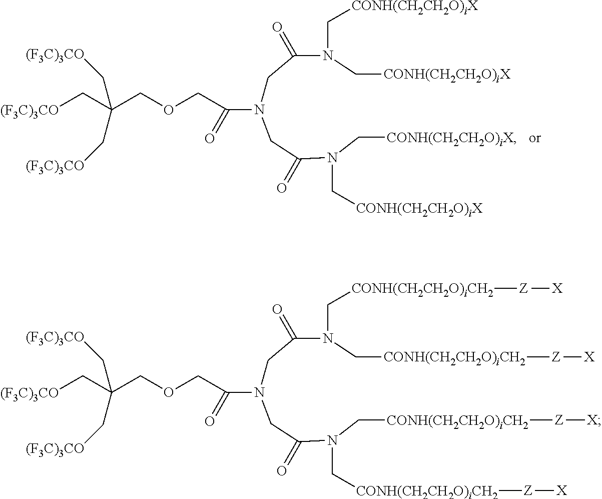
where i is a positive integer, each X is independently the chemotherapeutic agent or an ¹H contrast agent, and Z is a linker group.

17. The composition according to claim 16, wherein at least one X comprises:

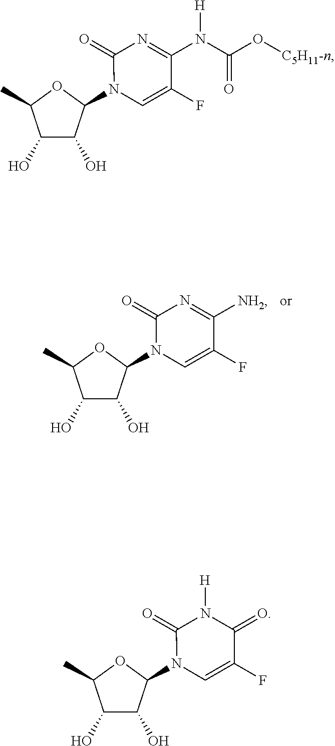

18. The composition according to claim 1, further comprising a pharmaceutically acceptable carrier.

19. A method of administering a drug treatment to a mammal, the method comprising:
administering to the mammal a dose of the composition of claim 1; and
measuring an amount of the composition in a tissue or organ of the mammal using ¹⁹F magnetic resonance imaging (MRI).

20. The method according claim 19, wherein the chemotherapeutic agent comprises a prodrug, and the imaging tracer is cleaved from the chemotherapeutic agent during conversion of the prodrug to an active drug, and further comprising:
administering to the mammal a plurality of doses of the composition;
intermittently conducting a plurality of measurements of the amount of the prodrug in the tissue or organ of the mammal using MRI;
determining an optimal dose of the prodrug or active drug for the mammal using the plurality of measurements; and
adjusting a dosage of the composition based upon one or more of the plurality of measurements.
